SEQUENCE LISTING (14 sequences)

SEQ ID NO: 1 - protein, 5 aa, synthetic construct
NYLIE

SEQ ID NO: 2 - protein, 17 aa, synthetic construct
VINPGHGFTNYNEKFKG

SEQ ID NO: 3 - protein, 9 aa, synthetic construct
GEWDWYFDV

SEQ ID NO: 4 - protein, 11 aa, synthetic construct
KASQNVGTAVA

SEQ ID NO: 5 - protein, 7 aa, synthetic construct
STSNRYT

SEQ ID NO: 6 - protein, 9 aa, synthetic construct
QQCSSYPFT

SEQ ID NO: 7 - protein, 17 aa, synthetic construct
VINPGHGFTNYAQKFQG

SEQ ID NO: 8 - protein, 9 aa, synthetic construct
QQSSSYPFT

SEQ ID NO: 9 - protein, 118 aa, synthetic construct
QVQLQQSGAELVRPGTSVKVSCKASGYAFTNYLIEWLKQRPGQGLEWIGVINPGHGFTNYNEKFKGKATLTADKSSSTAYMQLSSLTSDDSAVYFCARGEWDWYFDVWGAGTTVTVSS

SEQ ID NO: 10 - protein, 107 aa, synthetic construct
DIVMTQSQKFMSTSVGDRVSITCKASQNVGTAVAWYQQKPGQSPKLLIYSTSNRYTGVPDRFTGSGSGTDFTLTISNLQSEDLADYFCQQCSSYPFTFGSGTKLEIK

SEQ ID NO: 11 - protein, 118 aa, synthetic construct
QVQLVQSGAEVKKPGASVKVSCKASGYTFTNYLIEWVRQAPGQGLEWMGVINPGHGFTNYNEKFKGRVTMTRDTSTSTVYMELSSLRSEDTAVYYCARGEWDWYFDVWGQGTTVTVSS

SEQ ID NO: 12 - protein, 107 aa, synthetic construct
DIQLTQSPSFLSASVGDRVTITCKASQNVGTAVAWYQQKPGKAPKLLIYSTSNRYTGVPSRFSGSGSGTEFTLTISSLQPEDFATYYCQQCSSYPFTFGGGTKVEIK

SEQ ID NO: 13 - protein, 118 aa, synthetic construct
QVQLVQSGAEVKKPGASVKVSCKASGYTFTNYLIEWVRQAPGQGLEWMGVINPGHGFTNYAQKFQGRVTMTRDTSTSTVYMELSSLRSEDTAVYYCARGEWDWYFDVWGQGTTVTVSS

SEQ ID NO: 14 - protein, 107 aa, synthetic construct
DIQLTQSPSFLSASVGDRVTITCKASQNVGTAVAWYQQKPGKAPKLLIYSTSNRYTGVPSRFSGSGSGTEFTLTISSLQPEDFATYYCQQSSSYPFTFGGGTKVEIK

(19) United States
(12) Patent Application Publication
(10) Pub. No.: US 2026/0152555 A1
(43) Pub. Date: Jun. 4, 2026

(54) ANTI-CD112R ANTIBODIES AND USE THEREOF

(71) Applicant: HUAHUI HEALTH LTD., Beijing (CN)

(72) Inventors: Jianhua SUI, Beijing (CN); Yajing YANG, Beijing (CN); Fang YANG, Beijing (CN); Jianhe CHEN, Beijing (CN); Juan LIU, Beijing (CN); Yao SHENG, Beijing (CN); Chunmei LIU, Beijing (CN); Fangfang REN, Beijing (CN); Dongxia HAO, Beijing (CN); Xu HE, Beijing (CN)

(21) Appl. No.: 19/128,713

(22) PCT Filed: Sep. 22, 2023
(86) PCT No.: PCT/CN2023/120870

§ 371 Date: May 9, 2025

(30) Foreign Application Priority Data
Nov. 10, 2022 (WO) PCT/CN2022/131187

(51) Int. Cl. C07K 16/28; A61K 39/00; A61P 35/00

(52) U.S. Cl. CPC C07K 16/2803; A61P 35/00; A61K 2039/505; C07K 2317/24; C07K 2317/34; C07K 2317/732; C07K 2317/734; C07K 2317/76; C07K 2317/92

(57) ABSTRACT

Provided are CD112R-binding proteins, particularly anti-CD112R antibodies and antigen-binding fragments thereof. Further provided are compositions comprising the CD112R-binding proteins, nucleic acid molecule encoding the antigen-binding protein, and use thereof.

### FIELD OF THE INVENTION

The present disclosure relates to antibodies and fragment thereof for CD112R, as well as use thereof.

### BACKGROUND

Immunotherapies have emerged as extremely potent modalities for treating various cancers. One of the most impactful approaches is based on the blockade of immune checkpoint receptors or ligands.

CD112R (also called PVRIG, poliovirus receptor-related immunoglobulin domain-containing protein) preferentially expresses on T and NK cells and its expression is upregulated after cell activation. It competes with CD226 (a co-stimulatory receptor on CD8⁺ T cells and NK cells) for binding to their shared ligand CD112 with much higher affinity and then transduces inhibitory signal through its intracellular immunoreceptor tyrosine-based inhibitory motif (ITIM)-like motif, inhibiting immune functions of both T and NK cells.

In preclinical tumor models, the expression of CD112R and the CD112R-CD112 interaction are closely associated with tumor growth. Gene knockout (KO) or antibody blockade of CD112R inhibits tumor growth by enhancing the cytotoxic functions of tumor-infiltrating CD8⁺ T and NK cells, suggesting its potential as a target for cancer immunotherapy.

In cancer patients, CD112R is known to be upregulated and co-expressed with program cell death 1 (PD-1) and T cell immunoreceptor with Ig and ITIM domains (TIGIT) on tumor-infiltrating lymphocytes (TILs). Given that CD112 is highly expressed by tumor, epithelial, and myeloid cells in the tumor micro-environment (TME), it has been proposed that CD112R might inhibit antitumor immune responses via the CD112R-CD112 interaction. In addition, CD112R has a unique dominant expression on early memory (stem-like) T cell sub-population, which can self-renew and differentiate into effector cells, while CD112 is abundantly expressed across dendritic cells (DCs), resulting that the CD112R-CD112 interaction may inhibit T cell priming and expansion. Thus, CD112R blockade can enhance memory T cell activation by DCs, resulting in their increased expansion and differentiation. Additionally, dual blockade of CD112R and PD-1, or dual blockade of CD112R and TIGIT, further increases T cell activation and improves the effect of clinical treatment. These suggest that the CD112R-CD112 axis plays an important role in cancer immunity, and targeting this axis may help conquer the clinical limitations of immune checkpoint blockade (ICB) therapy.

### SUMMARY OF THE INVENTION

To overcome at least one of the above technical problems, the present disclosure provides monoclonal antibodies (mAbs) that targeted hCD112R and blocked its inhibitory function. These antibodies (Abs) were generated via hybridoma technology through immunizing mice with the extracellular domain (ECD) of hCD112R (hCD112R-ECD).

According to one aspect of the present disclosure, provided is antigen-binding protein which specifically binds to CD112R. In some embodiments, the antigen-binding protein can specifically bind to the extracellular domain (ECD) of CD112R.

In some embodiments, the antigen-binding protein can be an antibody or antigen-binding fragment thereof.

In some embodiments, the antigen-binding protein may comprise: (1) an immunoglobulin heavy chain variable region comprising a HCDR1 as set forth in SEQ ID NO: 1 or an amino acid sequence as set forth in SEQ ID NO: 1 with addition, deletion or substitution of one or more amino acid(s), a HCDR2 as set forth in SEQ ID NO: 2 or an amino acid sequence as set forth in SEQ ID NO: 2 with addition, deletion or substitution of one or more amino acid(s), and a HCDR3 as set forth in SEQ ID NO: 3 or an amino acid sequence as set forth in SEQ ID NO: 3 with addition, deletion or substitution of one or more amino acid(s); and (2) an immunoglobulin light chain variable region comprising a LCDR1 as set forth in SEQ ID NO: 4 or an amino acid sequence as set forth in SEQ ID NO: 4 with addition, deletion or substitution of one or more amino acid(s), a LCDR2 as set forth in SEQ ID NO: 5 or an amino acid sequence as set forth in SEQ ID NO: 5 with addition, deletion or substitution of one or more amino acid(s), and a LCDR3 as set forth in SEQ ID NO: 6 or an amino acid sequence as set forth in SEQ ID NO: 6 with addition, deletion or substitution of one or more amino acid(s).

It should be understood that the above addition, deletion or substitution of one or more amino acid(s) would not result in the loss of the binding ability of the antigen-binding protein to the CD112R.

In some embodiments, the antigen-binding protein may comprise: (1) an immunoglobulin heavy chain variable (VH) region comprising a HCDR1 as set forth in SEQ ID NO: 1, a HCDR2 as set forth in SEQ ID NO: 2, and a HCDR3 as set forth in SEQ ID NO: 3; and (2) an immunoglobulin light chain variable region comprising a LCDR1 as set forth in SEQ ID NO: 4, a LCDR2 as set forth in SEQ ID NO: 5, and a LCDR3 as set forth in SEQ ID NO: 6.

In some embodiments, the antigen-binding protein may comprise an immunoglobulin heavy chain variable (VH) region having an amino acid sequence as set forth in SEQ ID NO: 9 or an amino acid sequence with at least 85% sequence identity to the amino acid sequence as set forth in SEQ ID NO: 9. In some embodiments, the antigen-binding protein may comprise an immunoglobulin heavy chain variable (VH) region having an amino acid sequence with at least 85%, at least 90%, at least 95%, at least 98%, at least 99% or 100% sequence identity to the amino acid sequence as set forth in SEQ ID NO: 9.

In some embodiments, the antigen-binding protein may comprise an immunoglobulin light chain variable (VL) region having an amino acid sequence as set forth in SEQ ID NO: 10 or an amino acid sequence with at least 85% sequence identity to the amino acid sequence as set forth in SEQ ID NO: 10. In some embodiments, the antigen-binding protein may comprise an immunoglobulin light chain variable (VL) region having an amino acid sequence with at least 85%, at least 90%, at least 95%, at least 98%, at least 99% or 100% sequence identity to the amino acid sequence as set forth in SEQ ID NO: 10.

In some embodiments, the antigen-binding protein may be a humanized antibody or antigen-binding fragment thereof. In some embodiments, the humanized antibody or antigen-binding fragment thereof may comprise (1) a heavy chain variable (VH) region comprising a HCDR1 with an amino acid sequence as set forth in NYLIE, a HCDR2 with an amino acid sequence as set forth in VINPGHGFTNYX₁X₂KFX₃G, and a HCDR3 with an amino acid sequence as set forth in GEWDWYFDV; and/or (2) a light chain variable (VL) region comprising a LCDR1 with an amino acid sequence as set forth in KASQNVGTAVA, a LCDR2 with an amino acid sequence as set forth in STSNRYT, and a LCDR3 with an amino acid sequence as set forth in QQX₄SSYPFT. In some specific embodiments, X₁ may be selected from A (Ala) or N (Asn). In some specific embodiments, X₂ may be selected from E (Glu) or Q (Gln). In some specific embodiments, X₃ may be selected from K (Lys) or Q. In some specific embodiments, X₄ may be selected from C (Cys) or S (Ser).

In some specific embodiments, the humanized antibody or antigen-binding fragment thereof may comprise a heavy chain variable (VH) region comprising a HCDR1 with an amino acid sequence as set forth in SEQ ID NO:1, a HCDR2 with an amino acid sequence as set forth in SEQ ID NO: 2, and a HCDR3 with an amino acid sequence as set forth in SEQ ID NO: 3. In some specific embodiments, the humanized antibody or antigen-binding fragment thereof may comprise a heavy chain variable (VH) region comprising a HCDR1 with an amino acid sequence as set forth in SEQ ID NO: 1, a HCDR2 with an amino acid sequence as set forth in SEQ ID NO: 7, and a HCDR3 with an amino acid sequence as set forth in SEQ ID NO: 3.

In some specific embodiments, the humanized antibody or antigen-binding fragment thereof may comprise a light chain variable (VL) region comprising a LCDR1 with an amino acid sequence as set forth in SEQ ID NO: 4, a LCDR2 with an amino acid sequence as set forth in SEQ ID NO: 5, and a LCDR3 with an amino acid sequence as set forth in SEQ ID NO: 6. In some specific embodiments, the humanized antibody or antigen-binding fragment thereof may comprise a light chain variable (VL) region comprising a LCDR1 with an amino acid sequence as set forth in SEQ ID NO: 4, a LCDR2 with an amino acid sequence as set forth in SEQ ID NO: 5, and a LCDR3 with an amino acid sequence as set forth in SEQ ID NO: 8.

In some specific embodiments, the humanized antibody or antigen-binding fragment thereof may comprise (1) a heavy chain variable (VH) region comprising a HCDR1 with an amino acid sequence as set forth in SEQ ID NO:1, a HCDR2 with an amino acid sequence as set forth in SEQ ID NO: 2, and a HCDR3 with an amino acid sequence as set forth in SEQ ID NO: 3; and (2) a light chain variable (VL) region comprising a LCDR1 with an amino acid sequence as set forth in SEQ ID NO: 4, a LCDR2 with an amino acid sequence as set forth in SEQ ID NO: 5, and a LCDR3 with an amino acid sequence as set forth in SEQ ID NO: 6.

In some specific embodiments, the humanized antibody or antigen-binding fragment thereof may comprise (1) a heavy chain variable (VH) region comprising a HCDR1 with an amino acid sequence as set forth in SEQ ID NO:1, a HCDR2 with an amino acid sequence as set forth in SEQ ID NO: 7, and a HCDR3 with an amino acid sequence as set forth in SEQ ID NO: 3; and (2) a light chain variable (VL) region comprising a LCDR1 with an amino acid sequence as set forth in SEQ ID NO: 4, a LCDR2 with an amino acid sequence as set forth in SEQ ID NO: 5, and a LCDR3 with an amino acid sequence as set forth in SEQ ID NO: 8.

In some specific embodiments, the humanized antibody or antigen-binding fragment thereof may comprise a heavy chain variable (VH) region having an amino acid sequence as set forth in SEQ ID NO: 11 or an amino acid sequence with at least 85% sequence identity to the amino acid sequence as set forth in SEQ ID NO: 11. In some specific embodiments, the humanized antibody or antigen-binding fragment thereof may comprise a heavy chain variable (VH) region having an amino acid sequence as set forth in SEQ ID NO: 13 or an amino acid sequence with at least 85% sequence identity to the amino acid sequence as set forth in SEQ ID NO: 13. In some embodiments, the antibody or antigen-binding fragment thereof comprises a heavy chain variable (VH) region having an amino acid sequence with at least 85%, at least 90%, at least 95%, at least 98%, at least 99% or 100% sequence identity to the amino acid sequence as set forth in SEQ ID NO: 11 or 13.

In some specific embodiments, the humanized antibody or antigen-binding fragment thereof may comprise a light chain variable (VL) region having an amino acid sequence as set forth in SEQ ID NO: 12 or an amino acid sequence with at least 85% sequence identity to the amino acid sequence as set forth in SEQ ID NO: 12. In some specific embodiments, the humanized antibody or antigen-binding fragment thereof may comprise a light chain variable (VL) region having an amino acid sequence as set forth in SEQ ID NO: 14 or an amino acid sequence with at least 85% sequence identity to the amino acid sequence as set forth in SEQ ID NO: 14. In some embodiments, the antibody or antigen-binding fragment thereof comprises a light chain variable (VL) region having an amino acid sequence with at least 85%, at least 90%, at least 95%, at least 98%, at least 99% or 100% sequence identity to the amino acid sequence as set forth in SEQ ID NO: 12 or 14.

In some embodiments, the antigen-binding protein of the present disclosure can specifically bind to CD112R, particularly to the extracellular domain (ECD) of CD112R. In some embodiments, the antigen-binding protein can specifically bind to amino acids 90-150 of CD112R, particularly to amino acids 95-145 of CD112R. In some embodiments, the antigen-binding protein can particularly bind to R95, V90, W100, and/or E145 of CD112R. In some embodiments, the CD112R may be human CD112R and the above amino acid positions are based on human CD112R as set forth by UniProt accession #Q6DKI7.

In further embodiments, the antigen-binding protein may comprise a heavy chain constant region of IgG1 or IgG4 subtype, preferably IgG4 subtype.

In some embodiments, the antigen-binding fragment of the antibody may be a Fab, F(ab′)₂, Fv, or a single chain Fv fragment (scFv).

According to another aspect of the present disclosure, provided is a composition which comprises the antigen-binding protein of the present disclosure. In some embodiments, the composition may further comprise an additional therapeutic agent. In some embodiments, the composition may further comprise a pharmaceutically acceptable carrier.

According to yet another aspect of the present disclosure, provided is a nucleic acid molecule encoding the antigen-binding protein provided herein.

According to yet another aspect of the present disclosure, provided is a host cell which comprises the antigen-binding protein or the nucleic acid molecule of the present disclosure. In some embodiments, the host cell may be a prokaryotic or a eukaryotic cell. The host cell may be any kind of cellular system which can be engineered to generate the antibodies or fragments thereof according to the present disclosure. For example, the host cell may be an animal cell, in particular a mammalian cell. In specific embodiment HEK293 cells (human embryonal kidney cells), CHO (Chinese hamster ovary) cells or Vero cells can be used as host cells. In another embodiment, the host cell may be a non-human animal or mammalian cell, such as E. coli cells, Pichia cells.

According to yet another aspect of the present disclosure, provided is a method for prevention or treatment of a subject with a disease associated with CD112R, which comprises administrating the subject a therapeutically effective amount of the antigen-binding protein or the composition of the present disclosure.

According to yet another aspect of the present disclosure, provided is use of the antigen-binding protein or the composition of the present disclosure in the manufacture of a medicament for the prevention or treatment of a disease associated with CD112R.

According to yet another aspect of the present disclosure, provided is the antigen-binding protein or the composition of the present disclosure for use in the prevention or treatment of a disease associated with upregulated expression of CD112R.

In some embodiment, the disease may comprise cancer, an infectious disease, sepsis, an autoimmune condition, and/or an undesirable immune activation that follows gene therapy. In some embodiments, the disease may have upregulated level of CD112R in T and NK cells.

In some embodiments, the cancer may comprise, but be not limited to, carcinoma, lymphoma, blastoma, sarcoma, and leukemia. Particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), melanoma, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; multiple myeloma and post-transplant lymphoproliferative disorder (PTLD).

In some embodiments, the infectious disease may be chronic infections characterized by varying degrees of functional impairment of virus-specific T-cell responses, and this defect is a principal reason for the inability of the host to eliminate the persisting pathogen. Although functional effector T cells are initially generated during the early stages of infection, they gradually lose function during the course of the chronic infection as a result of persistent exposure to foreign antigen, giving rise to T cell exhaustion.

In some embodiments, the infectious disease may comprise infectious disorders, diseases and/or conditions, caused by a bacterial infection, viral infection, fungal infection and/or parasite infection.

According to yet another aspect of the present disclosure, provided is use of the antibodies or antigen-binding fragments thereof in the manufacture of a medicament for prevention or treatment of a subject with a disease associated with CD112R.

According to yet another aspect of the present disclosure, provided is According to yet another aspect of the present disclosure, provided is the antibodies or antigen-binding fragments thereof for use in prevention or treatment of a subject with a disease associated with CD112R.

The anti-CD112R antibodies or antigen-binding fragments provided by the present disclosure can effectively block the interaction between CD112R and CD112, and produce reverse lymphocyte functions such as cytotoxicity, and exert no complement dependent cytotoxicity (CDC) effector function and weak antibody dependent cellular cytotoxicity (ADCC) effector function.

### DESCRIPTION OF THE FIGURES

FIG. 1. Binding affinity and blocking activities of anti-hCD112R mAbs (mIgG2a form) analyzed by FACS. A) Binding analyses of anti-hCD112R mAbs or hCD112-ECD-mFc to YTS-hCD112R cells. Mouse anti-hCD112R mAbs at indicated concentrations were tested for binding to hCD112R-expressing YTS-hCD112R cells. B) Ligand competition analyses of mAbs. Mouse anti-hCD112R mAbs or hCD112-ECD-mFc at indicated concentrations were tested for blocking the binding of hCD112-ECD-hFc (0.6 μg/ml) to YTS-hCD112R cells by FACS-based competition assays. The hCD112-ECD-mFc protein was used as a control.

FIG. 2. Anti-hCD112R mAb 733 bound to hCD112R with high affinity. Multi-cycle kinetic analysis of the interaction between 733-mIgG2a and hCD112R using SPR.

FIG. 3. Anti-hCD112R mAb 733 efficiently blocked the human CD112R-CD112 interaction. Blocking activity of mAb 733 was analyzed by the FACS-based competition assay. 733-mIgG2a at serially diluted concentrations were tested for blocking the binding of hCD112-ECD-hFc to YTS-hCD112R cells.

FIG. 4. mAb 733 effectively reversed NK cell cytotoxicity suppressed by the human CD112R-CD112 interaction. A-B) The expression of hCD112R on YTS-hCD112R (A) or hCD112 on 721.221-hCD112 (B) stable cell line was assessed using anti-hCD112R Ab (Clone W16216D, Biolegend) or anti-hCD112 Ab (Clone TX31, Biolegend) by flow cytometry. C) The engagement of the CD112R-CD112 interaction disrupted the cytotoxicity of YTS-hCD112R cells (Left) and the presence of 733 effectively reversed the YTS-hCD112R cell cytotoxicity suppressed by the CD112R-CD112 interaction (right). For killing assays of 721.221 target cells by YTS cells and killing of 721.221-hCD112 cells by YTS-hCD112R cells, the ET ratio was 2:1.

FIG. 5. VH and VL sequence comparisons of mAb 733 and its humanized variants. A) VH amino acid sequence comparison. B) VL amino acid sequence comparison. Dots denote identical amino acids. The amino acid numbers are based on mAb 733 sequences. CDRs are determined according to the Kabat numbering system.

FIG. 6. Binding kinetics of mAb 733 and mAb 733-derived humanized Abs measured by SPR. A) Single-cycle kinetic analysis of the interaction between full-length mAb 733 or mAb 733-derived humanized Abs and hCD112R using SPR. B) Multi-cycle kinetic analysis of the interaction between full-length H733 Ab and hCD112R using SPR.

FIG. 7. Ligand competition activities of H733 analyzed by ELISA. H733 (hIgG4 form) at serially diluted concentrations were tested for competing with the hCD112-ECD-mFc for binding to the biotinylated hCD112R-ECD-His₆-Avi captured by immobilized streptavidin on an ELISA plate.

FIG. 8. Amino acid sequence alignment of N-terminal IgV domains of hCD112R and cynoCD112R. The amino acid numbers are based on hCD112R sequences. Dots denote identical amino acids.

FIG. 9. Mapping the binding epitope of H733 on hCD112R by human-to-cynomolgus mutation. A-B) Single-cycle kinetic analysis of the interaction between H733-hIgG4 (A) or hCD112-ECD-hFc (B) and hCD112R-ECD-mFc WT or variants using SPR.

FIG. 10. Mapping the binding epitope of H733 on hCD112R by using alanine-scanning mutagenesis. A) Sites selected for alanine-scanning mutagenesis on hCD112R according to the structure prediction by AlphaFold. B-C) Multi-cycle kinetic analysis of the interaction between H733-hIgG4 (B) or hCD112-ECD-hFc (C) and hCD112R-ECD-mFc WT or variants using SPR.

FIG. 11. H733 efficiently reversed CD112R-mediated NK cell dysfunction. YTS or YTS-hCD112R cells were co-cultured with 721.221-hCD112 cells at the ratio of 2:1, H733 or COM701 was added at indicated concentrations. LDH release was examined to evaluate the cytotoxicity. Here, the COM701 mAb, a humanized anti-hCD112R Ab developed by the Compugen Company, was used as a control. Both of H733 and COM701 are hIgG4 Abs.

FIG. 12. H733 augmented human T cell functions. A) The expression of the OKT3 Ab scFv fragment (up panel) and hCD112 (down panel) in CHO-OKT3scFv and CHO-OKT3scFv_hCD112 cell lines was assessed by flow cytometry using anti-mIgG Ab (Thermo Fisher) and anti-hCD112 Ab (Clone TX31, Biolegend), respectively. B) The expression of hCD112R in Jurkat reporter cells was assessed by flow cytometry using anti-hCD112R Ab (Clone W16216D, Biolegend). C) Jurkat-NFAT-luc-hCD112R reporter cells were co-cultured with CHO-OKT3-hCD112 cells. Serial dilutions of H733 or COM701 Abs (both are hIgG4 forms) were added. Relative luciferase units (RLU) were then recorded with a plate reader.

FIG. 13. H733 promoted T cell proliferation. Purified human T cells were labeled with CFSE and were co-cultured with CHO-OKT3 cells or CHO-OKT3_CD112 cells. H733 mAb (hIgG4 form) was included at the beginning of the culture. The proliferation of T cells was determined by CFSE dilution. Data are representative of triplicates.

FIG. 14. ADCC effector function induced by anti-hCD112R Abs. A) Raji-hCD112R cell line was generated for Fc-mediated effector functional analysis in vitro. The expression of hCD112R on Raji WT and Raji-hCD112R cells was analyzed by FACS using specific anti-hCD112R Ab (Clone W16216D, Biolegend). B) ADCC effector function induced by H733 Abs. ADCC activity was measured using a reporter assay system. Jurkat-NFAT-Luc2p/hFcγRIIIa (F158) were used as effector cells and Raji-hCD112R cells were used as target cells. Abs were tested at the indicated concentrations with three replicates. The E:T ratio was 6:1. Relative luminescence units (RLU) were plotted against the log Ab concentration. The luciferase activity is represented on the graphs.

FIG. 15. CDC effector function induced by H733 Abs. Raji-hCD112R target cells were incubated with H733 Abs in the presence of 5% rabbit complement sera. CDC activity was measured using Lactate dehydrogenase (LDH) release with three or four replicates. Abs were tested at 5 g/ml.

FIG. 16. H733 exhibited antitumor activity in xenogeneic mouse tumor models. A) The expression of hCD112 in MDA-MB-231, A375, and A549 tumor cell lines. Tumor cells from cell cultures were analyzed for the expression of hCD112 by FACS using a hCD112-specific Ab (Clone TX31, Biolegend). B) Antitumor activity of H733 in xenogeneic mouse tumor models. Black arrows indicate H733-hIgG4 treatment (10 mg/kg). Data are presented as mean values±SEM. (****P<0.0001)

### DETAILED DESCRIPTION OF THE INVENTION

Before the present methods and compositions are described, it is to be understood that this invention is not limited to a particular method or composition described and as such may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by those skilled in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

Cancer can be considered as an inability of the patient to recognize and eliminate cancerous cells. In many instances, these transformed (e.g. cancerous) cells counteract immunosurveillance. There are natural control mechanisms that limit T-cell activation in the body to prevent unrestrained T-cell activity, which can be exploited by cancerous cells to evade or suppress the immune response. Restoring the capacity of immune effector cells, especially T cells, to recognize and eliminate cancer is the goal of immunotherapy.

The field of immuno-oncology, sometimes referred to as “immunotherapy” is rapidly evolving, with several recent approvals of T cell checkpoint inhibitory antibodies. These antibodies are generally referred to as “checkpoint inhibitors” because they block normally negative regulators of T cell immunity. By inhibiting the checkpoint protein, for example through the use of antibodies that bind these proteins, an increased T cell response against tumors can be achieved. That is, these cancer checkpoint proteins suppress the immune response; when the proteins are blocked, for example using antibodies to against the checkpoint protein, the immune system is activated, leading to immune stimulation, resulting in treatment of conditions such as cancer and infectious disease.

Poliovirus Receptor-Related Immunoglobulin Domain-Containing Protein (PVRIG/CD112R) has recently been identified as an immune checkpoint molecule with potential for therapeutic development. PVRIG/CD112R is a single transmembrane protein consisting of a single extracellular IgV domain. In humans, PVRIG is expressed on T cells (predominantly CD8⁺ T cells) and natural killer (NK) cells, but not on B cells, monocytes or neutrophils. PVRIG binds to a single ligand, poliovirus receptor-related 2 (PVRL2, also known as CD112 or Nectin-2), and exerts an inhibitory effect on cytotoxic lymphocyte activity, likely via an ITIM-like motif in its intracellular domain. PVRL2 is an adhesion molecule involved in the formation of cell-cell junctions, and is overexpressed in various cancers. As PVRIG is present on both T cells and NK cells, blocking PVRIG provides the opportunity to augment both major cytotoxic effector cell types.

The present invention is directed to provide monoclonal antibodies specific for human CD112R or Poliovirus Receptor Related Immunoglobulin Domain Containing Protein (PVRIG). The monoclonal antibodies (mAbs) provided herein can efficiently block the inhibitory function of CD112R. These antibodies are specific for the CD112R extracellular domain.

It is noted that as used herein and in the appended claims, the singular forms “a,” “an,” and “the” include plural referents unless the context clearly dictates otherwise. Thus, for example, reference to “a recombinant AAV virion” includes a plurality of such virions and reference to “microglia” includes reference to one or more microglia cells and equivalents thereof known to those skilled in the art, and so forth.

The term “inhibition”, “inhibitor” or “antagonist” as used herein includes a reduction in a certain parameter, e.g., an activity, of a given molecule, e.g., an immune checkpoint inhibitor. For example, inhibition of an activity, e.g., CD112R activity, of at least 5%, 10%, 20%, 30%, 40% or more is included by this term.

The terms “anti-cancer effect” and “anti-tumor effect” can be used interchangeably herein. Both of the terms refer to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place.

The term “cancer” refers to a disease characterized by the rapid and uncontrolled growth of aberrant cells. Cancer cells can spread locally or through the bloodstream and lymphatic system to other parts of the body. Examples of various cancers are described herein and include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

Functional effects of CD112R-blocking antibodies on NK and T-cells can be assessed in vitro (and in some cases in vivo) by measuring changes in the following parameters: proliferation, cytokine release and cell-surface makers. For NK cells, increases in cell proliferation, cytotoxicity (ability to kill target cells), cytokine production (e.g. IFN-γ and TNF), or cell surface receptor expression (e.g. CD25) can be indicative of immune modulation, e.g. enhanced killing of cancer cells. For T-cells, increases in proliferation, cytotoxicity (ability to kill target cells), or cytokine production (e.g. IL-2, IL-4, IL-6, IFN-γ, TNF-α, IL-10, IL-17A) can be indicative of immune modulation, e.g. enhanced killing of cancer cells.

Accordingly, the present disclosure provides antibodies, including antigen binding fragments, that bind to human CD112R and methods of activating T cells and/or NK cells to treat diseases such as cancer and infectious diseases, and other conditions where increased immune activity results in treatment.

In the present disclosure, the numbering of the complementary determining regions (CDRs) is determined according to Kabat numbering system (Kabat et al., 1991, Sequences of Proteins of Immunological Interest, 5th Ed., United States Public Health Service, National Institutes of Health, Bethesda) which is entirely incorporated by reference.

The term “percent (%) sequence identity” with respect to an amino acid sequence is defined as the percentage of amino acid residues in a candidate sequence that are identical to the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

The term “humanized antibody” as used herein comprises one or more human framework regions in the variable region together with non-human (e.g., mouse, rat, or hamster) complementarity determining regions (CDRs) of the heavy and/or light chain. In some embodiments, a humanized antibody comprises sequences that are entirely derived from human except for the CDR regions. Humanized antibodies are typically less immunogenic to humans, relative to non-humanized antibodies, and thus offer therapeutic benefits in certain situations.

The anti-CD112R antibodies or antigen-binding fragments of the present disclosure can be used for treating patients, such as human subjects, generally with a condition associated with CD112R. The term “treatment” as used herein, refers to both therapeutic treatment and prophylactic or preventative measures, which in this example relates to treatment of cancer, as well as infectious disease, sepsis, and/or autoimmune conditions, and/or for inhibiting an undesirable immune activation that follows gene therapy. Those in need of treatment include those already with cancer as well as those in which the cancer is to be prevented. Hence, the mammal to be treated herein may have been diagnosed as having the cancer or may be predisposed or susceptible to the cancer. As used herein the term “treating”, “treat” or “treatment” refers to preventing, delaying the onset of, curing, reversing, attenuating, alleviating, minimizing, suppressing, halting the deleterious effects or stabilizing of discernible symptoms of the above-described cancerous diseases, disorders or conditions. It also includes managing the cancer as described above. By “manage” it means reducing the severity of the disease, reducing the frequency of episodes of the disease, reducing the duration of such episodes, reducing the severity of such episodes, slowing/reducing cancer cell growth or proliferation, slowing progression of at least one symptom, amelioration of at least one measurable physical parameter and the like.

The term “Fab” or “Fab region” as used herein refers to the polypeptide that comprise the VH, CH1, VL and CL immunoglobulin domains. Fab may refer to this region in isolation, or this region in the context of a full length antibody, antibody fragment or Fab fusion protein.

As used herein the term “infectious disorder and/or disease” and/or “infection”, used interchangeably, includes any disorder, disease and/or condition caused by presence and/or growth of pathogenic biological agent in an individual host organism. As used herein the term “infection” comprises the disorder, disease and/or condition as above, exhibiting clinically evident illness (i.e., characteristic medical signs and/or symptoms of disease) and/or which is asymptomatic for much or all of it course. As used herein the term “infection” also comprises disorder, disease and/or condition caused by persistence of foreign antigen that lead to exhaustion T cell phenotype characterized by impaired functionality which is manifested as reduced proliferation and cytokine production.

The term “sepsis” as used in herein encompasses Sepsis, Severe sepsis. Septic shock, Systemic inflammatory response syndrome (SIRS), Bacteremia, Septicemia, Toxemia, Septic syndrome.

The term “CDC” or “complement dependent cytotoxicity” as used herein refers to one mechanism by which antibodies can mediate specific target cell lysis through activation of an organism's complement system. The term “ADCC” or “antibody dependent cellular cytotoxicity” as used herein refers to the killing of a target cell which is coated with the antibodies by an effector cell of the immune system. Both CDC and ADCC can contribute to Ab-induced cell lysis.

Unless otherwise defined, all scientific and technical terms used herein have the same meaning as commonly understood by those skilled in the art to which this technology belongs.

The following examples are set forth to provide the ordinarily skilled artisan with a complete disclosure and description for guidance as to how to produce the antibodies and fragments disclosed herein, and are not intended to limit the scope of the invention disclosed herein. In addition, the following examples are not intended to represent that the experiments below are all or the only experiments.

### EXAMPLES

### Example 1. Generation of hCD112R Blocking mAbs by Hybridoma Technology

### 1.1 Methods

### Human CD112R Recombinant Proteins for Mouse Immunization and ELISA-Based Binding and Competition Assays

The extracellular domain (ECD) consisting of amino acids (AA) 42-172 of human CD112R (hCD112R, Uniprot accession #Q6DKI7) (hCD112R-ECD) was amplified by PCR and cloned in an expression vector with C-terminus fused either to a His₆-Avi tag (hCD112R-ECD-His₆-Avi) or to the Fc domain of mouse IgG2a (hCD112R-ECD-mFc). These fusion proteins were expressed in HEK 293F cells by transient transfection and then purified by affinity chromatography.

### Mouse Immunization and Hybridoma Fusion

Six-week-old BALB/c mice were immunized subcutaneously with 100 μl of adjuvant (Sigma-Aldrich) containing 50 μg hCD112R-ECD-mFc. The immunization was conducted by two to three injections of the above immunogen with three weeks apart. Blood samples were collected 1 week after each immunization by tail bleeding. Mouse sera were determined for reactivity to hCD112R-ECD by ELISA (enzyme-linked immunosorbent assay)-based binding assays. Animals with highest anti-hCD112R Ab titers in sera were selected and boosted intraperitoneally with 50 μg of hCD112R-ECD-mFc in the absence of any adjuvant. Three days after boosting, the splenocytes were isolated and fused with the murine myeloma cell line, SP2/0 cells, using conventional techniques. The supernatants of hybridoma clones were screened by ELISA-based binding and competition assays.

Amplification of Functional Immunoglobulin Variable Genes from Hybridomas and Construction of Full-Length Mouse IgG2a (mIgG2a) Abs

VH and VL sequences of mAb 733 were amplified using cDNA from hybridomas with both binding and competition activities in ELISA-based assays. The PCR product of VH and VL genes was directly cloned into a TA cloning vector using a pMD™ 18-T Vector Cloning Kit (Takara). TA plasmids containing inserted VH and VL genes were sequenced and analyzed using the IgBLAST tool. Then these VH and VL PCR products were cloned into expression vectors that contained the constant regions of heavy (CH) and light (CL) chains of the mIgG2a molecule, respectively. The VH and VL sequences of mAb 733 encode amino acid sequences as shown by SEQ ID NOs: 9 and 10, respectively.

Preparation of full-length mIgG2a Abs

To produce full-length mIgG2a Abs, HEK 293F (Life Technologies) cells were transiently co-transfected with the two expression plasmids (heavy chain+light chain plasmids) at a 1:1 ratio. 3 to 6 days after transfection, the cell culture supernatant was harvested for purification of IgG Ab by Protein A affinity chromatography (Protein A Sepharose CL-4B, GE Healthcare).

### ELISA-Based Binding and Competition Assays

For the ELISA-based binding assays, biotinylated hCD112R-ECD-His₆-Avi protein was captured with streptavidin (Sigma) coated 96-well plates (Nunc, MaxiSorp™). For cell culture supernatant-based ELISA, the supernatants diluted in 2% milk/PBS were added, and then detected by HRP-anti-mouse IgG secondary Ab (Thermo Fisher Scientific). For mouse serum-based ELISA, the mouse serum diluted in 2% milk/PBS was added, and then detected by HRP-anti-mouse IgG secondary Ab (Thermo Fisher Scientific). For full-length mouse IgG-based ELISA, IgG Abs diluted in 2% milk/PBS were added, and the bound Abs were detected using an HRP-anti-mouse IgG secondary Ab (Thermo Fisher Scientific).

The ELISA-based competition assays were performed in a manner similar to ELISA-based binding assays, except that tested Abs were incubated with captured antigens in the presence of competitive ligand. Briefly, different Abs serially diluted in 2% milk/PBS containing 0.02 μg/ml of the extracellular domain of hCD112 fused to the Fc domain of human IgG1 (hCD112-ECD-hFc) were added to the ELISA plates to test for competitive binding between hCD112R and hCD112. The signal was measured via ligand detection using HRP-anti-human IgG secondary Ab (Thermo Fisher Scientific).

### FACS-Based Binding and Competition Assays

YTS cell line stably expressing the full length of hCD112R (YTS-hCD112R) was used in this assay. For cell line construction, an expression plasmid was constructed by inserting the full-length hCD112R cDNA into the vector. The expression plasmid was then transfected into YTS cells using the Nucleofector transfection system (Lonza, Nucleofector kit V), followed by FACS sorting of hCD112-staining positive populations. Sorted positive cells were cultured under the selection of G418.

For FACS-based binding assay, YTS-hCD112R cells were incubated with different anti-hCD112R mAbs or hCD112-ECD-mFc fusion protein serially diluted in 0.5% BSA/PBS at 4° C. for 1 h. Then cells were washed three times with 0.5% BSA/PBS. Abs or ligand binding to cells were detected by adding goat anti-mouse IgG-FITC Ab (Pierce-Thermo Fisher Scientific).

For FACS-based competition assay, YTS-hCD112R cells were incubated with different Abs in mIgG2a format (60 or 0.6 g/ml) in the presence of competitive ligand hCD112-ECD-hFc at 0.6 μg/ml at 4° C. for 1 h. Then cells were washed three times with PBS containing 0.5% BSA. Ligands binding to cells were detected by adding goat anti-human IgG-FITC Ab (Pierce-Thermo Fisher Scientific).

### 1.2 Results

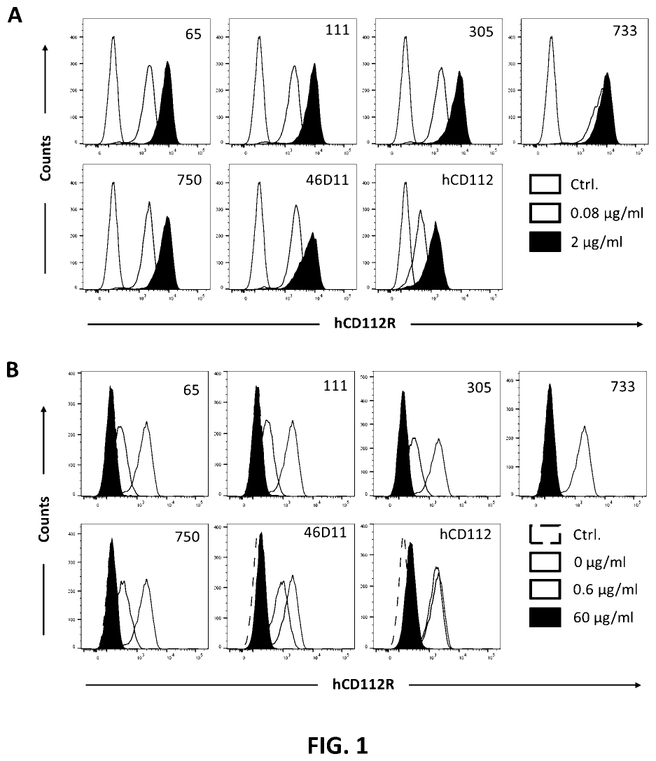

Anti-hCD112R mAbs were generated based on conventional hybridoma fusion technology. mAbs with high binding activities in ELISA-based binding assays and strong competition activities in ELISA-based competition assays were selected for further characterization. The binding affinity (FIG. 1A) and blocking activities (FIG. 1B) of these mAbs were tested by FACS-based binding assays and competition assays, respectively. The results indicated that mAb 733 represented higher binding affinity and stronger blocking activity as compared with other mAbs. Therefore, mAb 733 was selected for further characterization.

### Example 2. Anti-hCD112R mAb 733 Bound to hCD112R with High Affinity and Efficiently Blocked the Human CD112R-CD112 Interaction

### 2.1 Methods

Multi-Cycle Kinetic Analysis of the Interaction Between 733 and hCD112R by SPR.

Kinetic analyses of the binding of mAb 733 to the hCD112R-ECD were performed on a Biacore T200 instrument (Biacore, GE Healthcare). Protein A/G (Thermo Fisher) was covalently attached to surfaces of a CM5 sensor chip using an amine coupling kit (GE Healthcare). 733-mIgG2a (2 μg/ml) was captured on the chip and the analytes (hCD112R-ECD-His₆-Avi) at serial dilutions were then injected. Binding kinetics were evaluated using a 1:1 Langmuir binding model. The association rates (ka), dissociation rates (kd), and affinity constants (KD) were calculated using Biacore T200 evaluation software.

### FACS-Based Competition Assay

For the FACS-based competition assay, YTS-hCD112R cells were incubated with serially diluted 733-mIgG2a in the presence of competitive ligand (hCD112-ECD-hFc) at 4° C. for 30 min. Then cells were washed three times with PBS containing 0.5% BSA. Ligands binding to cells were detected by adding goat anti-human IgG-FITC Ab (Pierce-Thermo Fisher Scientific).

### 2.2 Results

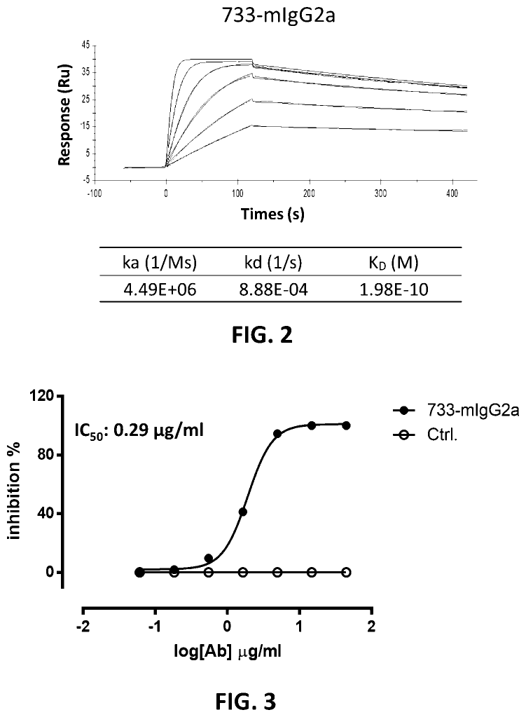

To examine the binding kinetic constant of mAb 733 in real time binding reactions, surface plasmon resonance (SPR) measurement was performed. The result showed that mAb 733 bound to hCD112R with high affinity (K_{D}=0.2 nM) (FIG. 2). To determine the competition activity of mAb 733, FACS-based competition assay was performed. mAb 733 efficiently blocked the human CD112R-CD112 interaction, with an IC₅₀ value of 0.29 μg/ml (FIG. 3).

### Example 3. Anti-hCD112R mAb 733 Effectively Reversed the NK Cell Cytotoxicity Suppressed by the Human CD112R-CD112 Interaction

### 3.1 Methods

### NK Cell Cytotoxicity Assay

1) Establishment of YTS-hCD112R and 721.221-hCD112 Stable Cell Lines.

The generation of YTS-hCD112R stable cell line has been described before. For the generation of 721.221-hCD112 stable cell line, full-length cDNA of hCD112 delta was amplified by PCR and cloned into a mammalian cell expression plasmid. 721.221 cells were then transfected with full-length hCD112 expressing plasmid using Nucleofector transfection system (Lonza, Nucleofector kit V) following the manufacturer's instruction. One day after transfection, the transfected cells were immunostained with the anti-hCD112 Ab (Clone TX31, Biolegend), and then positive cells were enriched by FACS sorting. The sorted positive cells were cultured under the selection of puromycin.

### 2) Cell Cytotoxicity Assay

721.221-hCD112 target cells (10000 cells/well) were incubated with YTS-hCD112R effector cells at the effector-to-target (E:T) ratio of 2:1 without or with adding mAb 733 at a final concentration of 10 μg/ml for 6 h. Then lactate dehydrogenase (LDH) released by cells was detected following the instruction of a CytoTox 96® Non-Radioactive Cytotoxicity Assay kit (Promega). Cytotoxicity percentages were calculated following the manufacturer's instruction.

### 3.2 Results

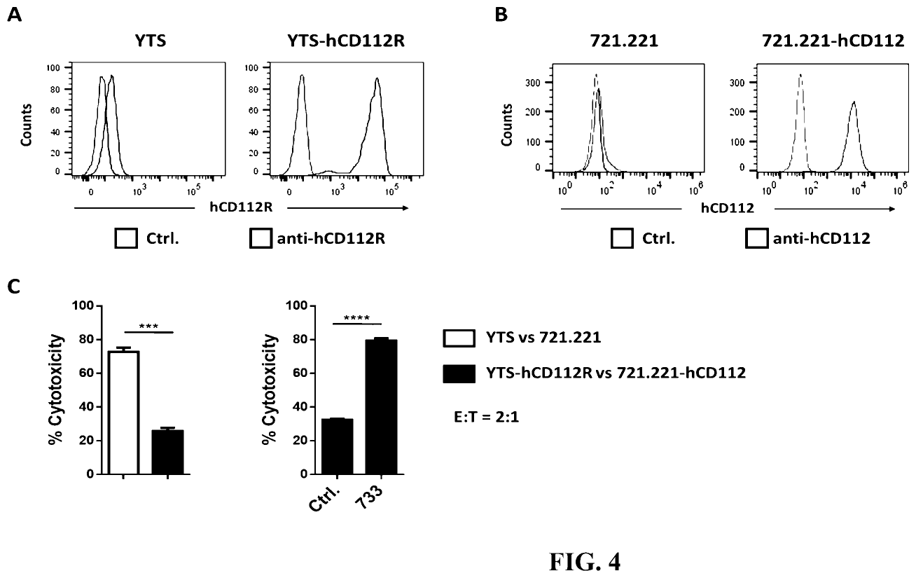

In this example, mAb 733 was investigated whether it could restore immune cell activities by blocking the human CD112R-CD112 interaction. It has been known that CD112R was upregulated on T/NK cells and inhibited T/NK cell-mediated cytotoxicity through interaction with CD112 in cancers. We then evaluated whether mAb 733's binding with hCD112R could interrupt the human CD112R-CD112 interaction-mediated inhibitory functions on NK cells. Previous studies showed that YTS cells (an NK cell line) achieved restricted killing of 721.221 target cells (an MHC class I-negative human B cell line). Here, we established YTS cells stably expressing hCD112R (YTS-hCD112R) and 721.221 cells stably expressing hCD112 (721.221-hCD112) (FIG. 4A-B). YTS cells-mediated cytotoxicity to 721.221 cells can be effectively inhibited by the human CD112R-CD112 interaction, whereas mAb 733 significantly restored the ability of YTS-hCD112R cells to kill 721.221-hCD112 cells (FIG. 4C). This result demonstrated that mAb 733 efficiently blocked the human CD112R-CD112 interaction-mediated inhibitory functions.

### Example 4. Humanization of mAb 733

### 4.1 Methods

### Humanization of mAb 733

For the humanization of mAb 733, amino acid sequences of human germline immunoglobulin G (IgG) that are homologous to the amino acid sequences of the mAb 733 were searched by blasting the human immunoglobulin gene database in NCBI website (http://www.ncbi.nlm.nih.gov/igblast/). The human frameworks with highest homology to the framework of mAb 733 were selected as the template for CDR grafting. Additional mutations were made to assess the impact on the Ab binding.

Kinetic Analysis of the Interaction Between mAb 733 and 733-Derived Humanized Abs to hCD112R by SPR

Kinetic analyses of the bindings of 733 and various 733-derived humanized Abs to the hCD112R-ECD protein were performed using a Biacore T200 instrument (Biacore, GE Healthcare). Anti-hFc Ab (Thermo Fisher) was covalently attached to surfaces of a CM5 sensor chip using an amine coupling kit (GE Healthcare). Abs at optimal concentrations were captured on the chip and the analytes (hCD112R-ECD-His₆-Avi) at single concentration or serial dilutions were then injected. Binding kinetics were evaluated using a 1:1 Langmuir binding model. The association rates (ka), dissociation rates (kd), and affinity constants (KD) were calculated using Biacore T200 evaluation software.

### ELISA-Based Competition Assays

Biotinylated hCD112R-ECD-His₆-Avi protein antigen was captured with streptavidin (Sigma) coated 96-well plates (Nunc, MaxiSorp™). H733 serially diluted in 2% milk/PBS containing 0.1 μg/ml of the hCD112-ECD-mFc protein were added to the ELISA plates to test for competitive binding between hCD112R and hCD112. The signal was measured via ligand detection using HRP-anti-mouse IgG secondary Ab (Thermo Fisher Scientific).

### 4.2 Results

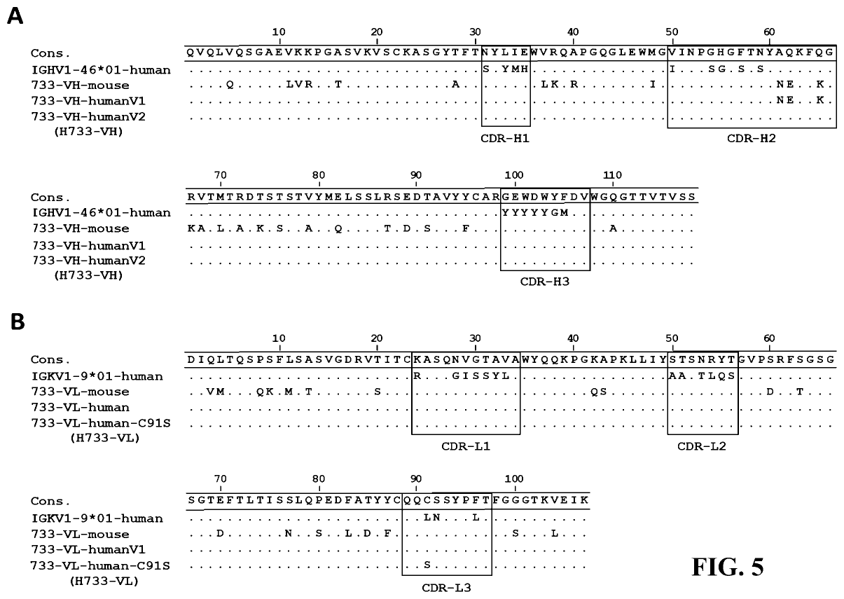

For humanization of the mAb 733, we searched human germline IgG domain sequences homologous to the amino acid sequences of the mAb 733 by blasting the human immunoglobulin database in NCBI website (http://www.ncbi.nlm.nih.gov/igblast/). The human frameworks with highest homology to the framework of mAb 733 were selected as the template for CDR grafting. FIG. 5 showed that the Kabat-defined CDRs of mAb 733 heavy chain (SEQ ID NO: 9) and light chain (SEQ ID NO: 10) were grafted into the closest human germline templates IGHV1-46*01 and IGKV1-9*01, respectively. The VL and VH chains of mAb 733 after grafting were named as Ab 733-VL-humanV1 (SEQ ID NO: 12) and Ab 733-VH-humanV1 (SEQ ID NO: 11). Given that the unpaired cysteine in VL-CDR3 domain of the mAb 733 may affect protein expression level, the cysteine was further changed to serine (FIG. 5). This humanized VL with one amino acid substitution was named as Ab 733-VL-human-C91S (SEQ ID NO: 14). In addition, three amino acids in CDR2 of Ab 733-VH-humanV1 were further mutated from the original mouse residues to the human germline residues. This resulting VH was named as Ab 733-VH-humanV2 (SEQ ID NO: 13). Full length IgG Abs composed of various VH/VL combinations were expressed, and their binding affinities to hCD112R were analyzed by SPR.

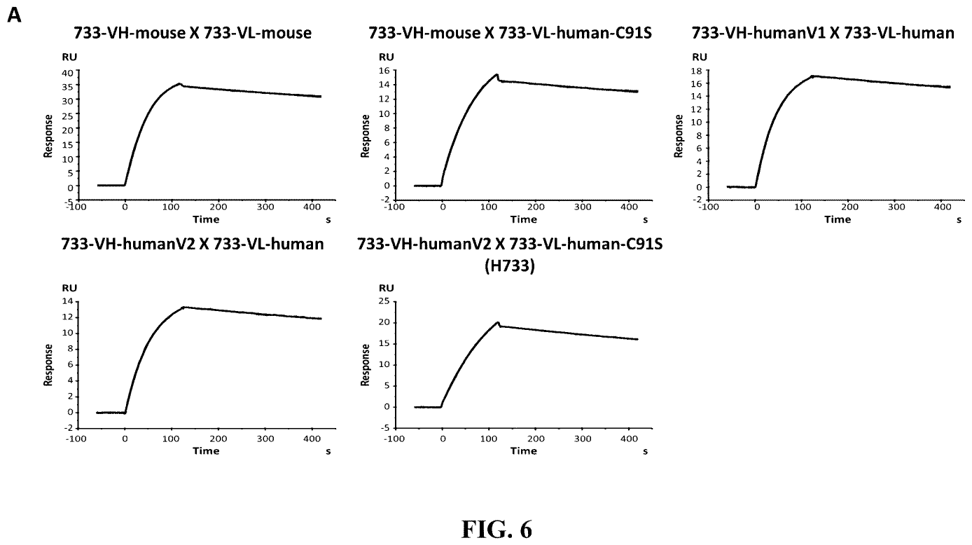

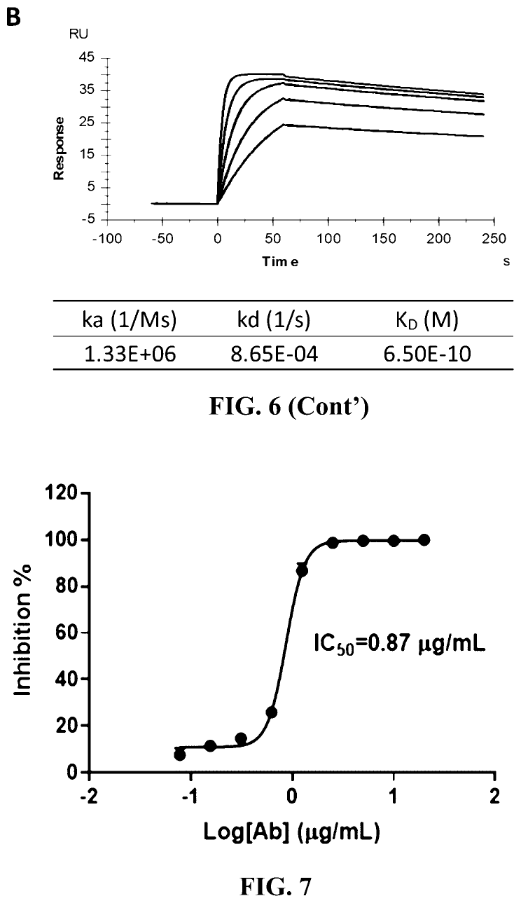

The result showed that the binding affinities of different humanized Ab versions were almost the same as that of parental mAb 733 (FIG. 6A). Ab composed of mAb 733-VH-humanV2 and 733-VL-human-C91S was named as H733. The binding affinity of H733 to hCD112R was further evaluated by multi-cycle kinetic analysis. The result showed that the binding affinity of H733 (KD=0.65 nM) was comparable to that of 733 (FIG. 6B). The blocking activity of H733 was tested by ELISA-based competition assay. The result indicated that H733 could efficiently block the CD112R-CD112 interaction, with an IC₅₀ value of 0.87 μg/ml (FIG. 7). Thus, H733 was used for following experiments.

### Example 5. Epitope Mapping of H733

### 5.1 Methods

Kinetic Analysis of the Interaction Between H733 or hCD112 and Mutated hCD112R by SPR.

Kinetic analyses of the bindings of WT or mutated hCD112R-ECD-mFc proteins to H733 and hCD112-ECD were performed on a Biacore T200 instrument (Biacore, GE Healthcare). For the single-cycle SPR analysis, anti-mFc Ab (Thermo Fisher) was covalently attached to surfaces of a CM5 sensor chip using an amine coupling kit (GE Healthcare). WT or mutated hCD112R-ECD mFc fusion proteins were captured on the chip and H733-hIgG4 or hCD112-ECD-hFc were then injected. For the multi-cycle SPR analysis, anti-hFc Ab (Thermo Fisher) was covalently attached to surfaces of a CM5 sensor chip using an amine coupling kit (GE Healthcare). H733-hIgG4 or hCD112-ECD-hFc protein was captured on the chip and 2-fold serially diluted WT or mutated hCD112R-ECD-mFc proteins (3.125-100 nM) were then injected. Binding kinetics were evaluated using a 1:1 Langmuir binding model. The association rates (ka), dissociation rates (kd), and affinity constants (KD) were calculated using Biacore T200 evaluation software.

### 5.2 Results

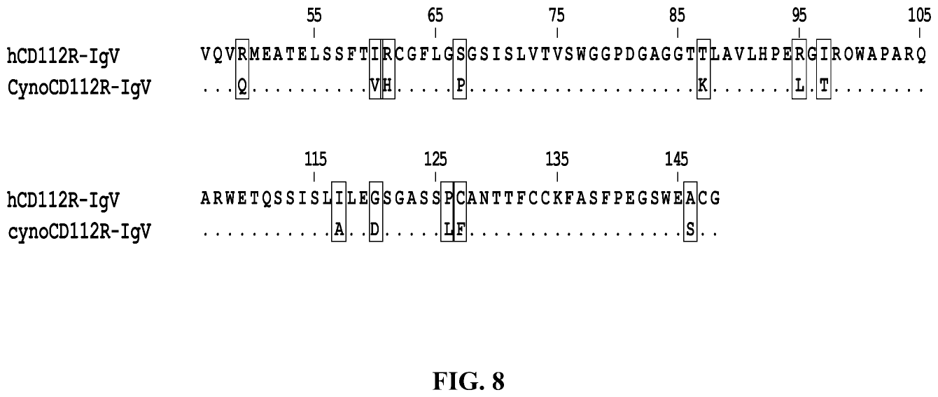

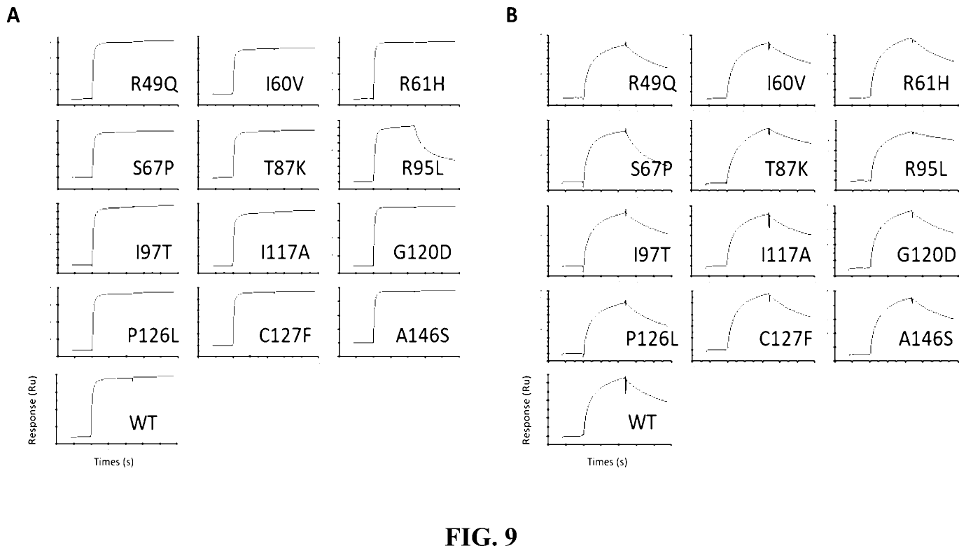

To investigate the sites on hCD112R that may contribute to H733's binding, we constructed several hCD112R variants by replacing residues in the IgV domain of hCD112R with the corresponding residues from cynomolgus CD112R (cynoCD112R) (FIG. 8). These hCD112R variants and wildtype (WT) hCD112R protein were expressed and purified, and then were tested for binding to both H733 and hCD112 ligand by SPR. Results of single-cycle SPR analysis showed that R95L human-to-cyno mutation of CD112R negatively impacted H733's binding (FIG. 9).

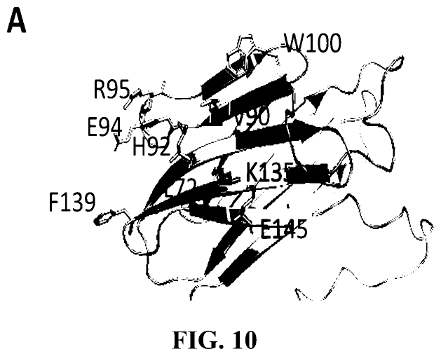

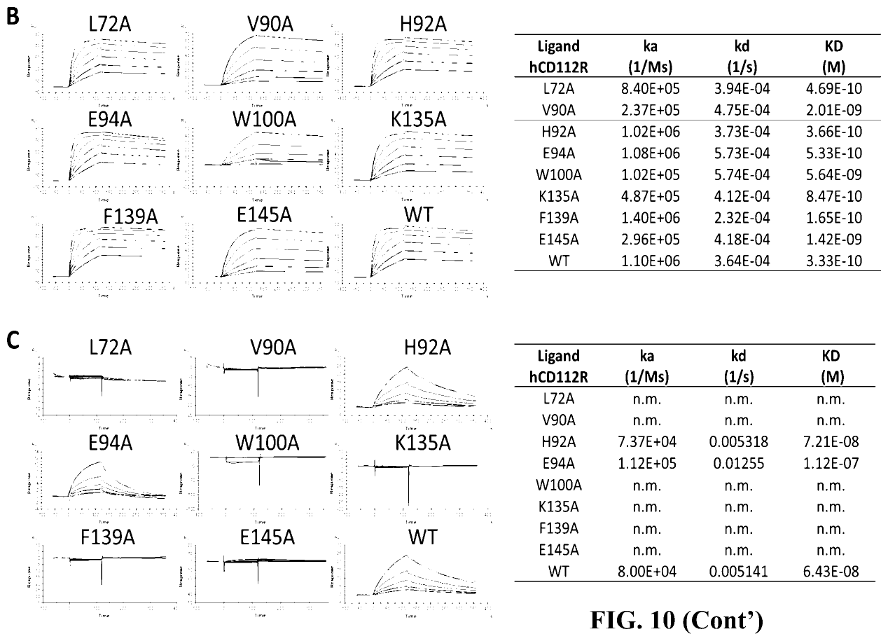

According to the above results, we next selected other residues of the hCD112R-IgV domain for the alanine-scanning mutagenesis based on the structure predicted using AlphaFold Protein Structure Database. These residues were L72, V90, H92, E94, W100, K135, F139, and E145 (FIG. 10A). After expression and purification, these hCD112R variants were used to analyze the binding to H733 and hCD112. Results of Multi-cycle SPR showed that V90A, W100A, or E145A mutation of hCD112R reduced the binding to H733 (about 4-15-fold decrease as compared with WT hCD112R) (FIG. 10B). Notably, V90A, W100A, or E145A mutation of hCD112R also dramatically reduced the binding to hCD112 (FIG. 10C). Taking together, by applying site-directed mutagenesis mapping, we found that R95, V90. W100, and E145 residues of hCD112R impacted the binding to H733.

### Example 6. H733 Efficiently Reversed the Human Lymphocyte Functions Inhibited by the CD112R-CD112 Interaction

### 6.1 Methods

### NK Cell Cytotoxicity Assay

721.221-hCD112 cells (10000 cells/well) were incubated with YTS-hCD112R cells at the indicated E:T ratio of 2:1 at the presence of serially diluted anti-hCD112R mAbs for 6 h. Then lactate dehydrogenase (LDH) released by cells was detected following the instruction of a CytoTox 96® Non-Radioactive Cytotoxicity Assay kit (Promega). Cytotoxicity percentages were calculated following the manufacturer's instruction.

### Human T Cell Proliferation Assay

### 1) Isolation of Primary Human T Cells

Human peripheral blood mononuclear cells (PBMCs) from healthy donors were purchased from the ORICELLS. Human T cells were negatively selected and purified by the EasySep human T cell enrichment kit (STEMCELL, #19051) according to the manufacturer's instruction.

### 2) Construction of Human T Cell Stimulator Cells

CHO cells were transfected with expressing plasmid that codes the membrane-bound anti-human CD3 mAb OKT3 single-chain variable fragment (OKT3-scFv) sequence (Leitner et al., Journal of immunological methods (2010) 362, 131-141) to obtain a pool of CHO cells expressing membrane-bound OKT3-scFv. After 24 h of transfection, cells were stained with FITC conjugated goat anti-mouse whole IgG Ab (Sigma Aldrich), and single cells with positive staining were isolated by FACS single cell sorting and cultured with 200 μl complete culture medium containing G418. To test the effect of H733 on the human T cell proliferation, CHO-OKT3-scFv stable cell line was transfected with expressing plasmid that codes the hCD112 delta full-length molecule (CHO-OKT3-scFv-hCD112). After 24 h of transfection, cells were stained with APC conjugated anti-hCD112 Ab (Clone TX31, Biolegend). Positive cells were then enriched by FACS sorting.

### 3) Analyses of Human T Cell Proliferation

Human T cells enriched by negative selection were labeled with CFSE and stimulated with stimulator cells. Stimulator cells (CHO-OKT3-scFv or CHO-OKT3-scFv-hCD112) were treated with mitomycin C (10 μg/ml for 10 h) before being co-cultured with CFSE-labeled human T cells at the ratio of 1:100. Control Ab or H733 were added at 1 μg/ml at the beginning of the culture. T cell proliferation was assessed by CFSE dilution after a 5-day culture.

### Luciferase Reporter Assay

### 1). Establishment of Jurkat-NFAT-luc_hCD112R Cell Line

Jurkat-NFAT-luc-hCD112R cells were generated by electroporating the plasmid coding full-length hCD112R cDNA sequence into Jurkat-NFAT-luc cells (purchased from InvivoGen) and selected with G418 antibiotic (500 μg/ml). Surface expression of hCD112R was confirmed using flow cytometry after staining with PE labeled anti-hCD112R Ab (Clone301503, Biolegend).

### 2). Luciferase Reporter Assay

The CHO-OKT3scFv or CHO-OKT3scFv-hCD112 cells were harvested and seeded into a 96-well flat plate at 50,000 cells in 100 μL of the culture medium (DMEM with 10% FBS) per well, followed by incubation at 37° C. with 5% CO₂ for 2 h. Then, the culture medium was removed and Jurkat-NFAT-luc-hCD112R cells were added at 50,000 cells in 100 μl of assay medium (IMDM with 10% FBS) per well. The anti-hCD112R Abs were serially diluted at a ratio of 1:2 in the assay medium from a starting concentration of 5 μg/ml and then were added into each well. After that, the plate was incubated at 37° C. in 5% CO₂ for 18 h. Then, 20 μL of sample per well was pipetted into a 96-well black plate and 50 μL of QUANTI-Luc™ assay solution was added to each well. Relative luciferase units (RLU) were then recorded with a plate reader (BioTek Synergy HIM).

### 6.2 Results

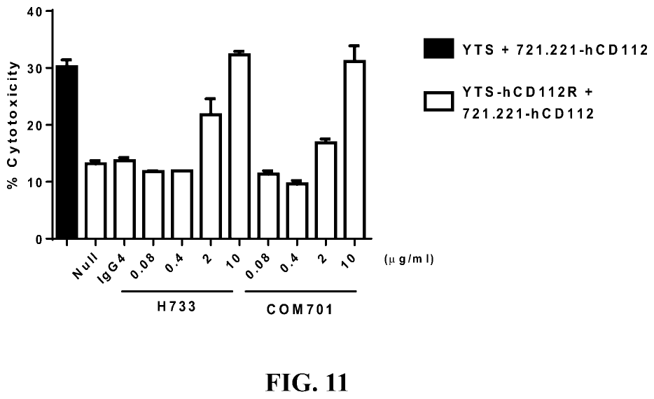

We evaluated the blocking function of H733 on NK cells using YTS-hCD112R/721.221-hCD112 cell killing assay. Here, an Ab named COM701 (US Patent No.: US20160244521A1), which was developed by the Compugen Company, was used as an anti-hCD112R reference Ab control. The result showed that both of H733 and COM701 efficiently reversed the CD112R-mediated NK cell dysfunction (FIG. 11).

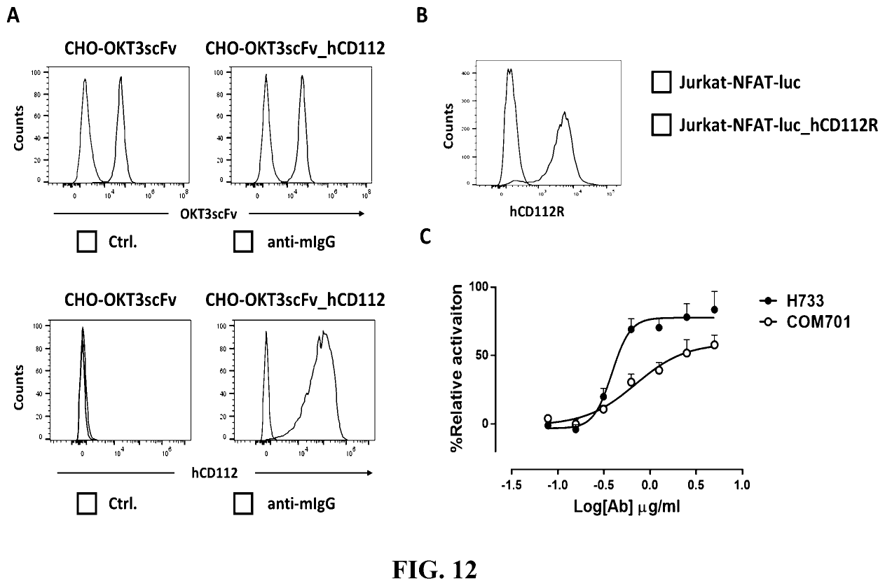

We next tested the potential function of H733 on human T cells using the Jurkat luciferase reporter assay and the proliferation assay. As for the Jurkat luciferase reporter assay, CHO cells engineered to express membrane-bound anti-human CD3 Ab fragment (CHO-OKT3scFv) or express both membrane-bound OKT3scFv fragment and hCD112 (CHO-OKT3scFv_hCD112) (FIG. 12A) were incubated with Jurkat-NFAT-luc-hCD112R reporter cells (FIG. 12B). The addition of H733 or COM701 increased luciferase production (FIG. 12C), with EC₅₀ values of 0.39 μg/ml and 0.69 μg/ml, respectively, suggesting that blockade of the CD112R-CD112 interaction by H733 can enhance human T cell pro-inflammatory cytokine signaling.

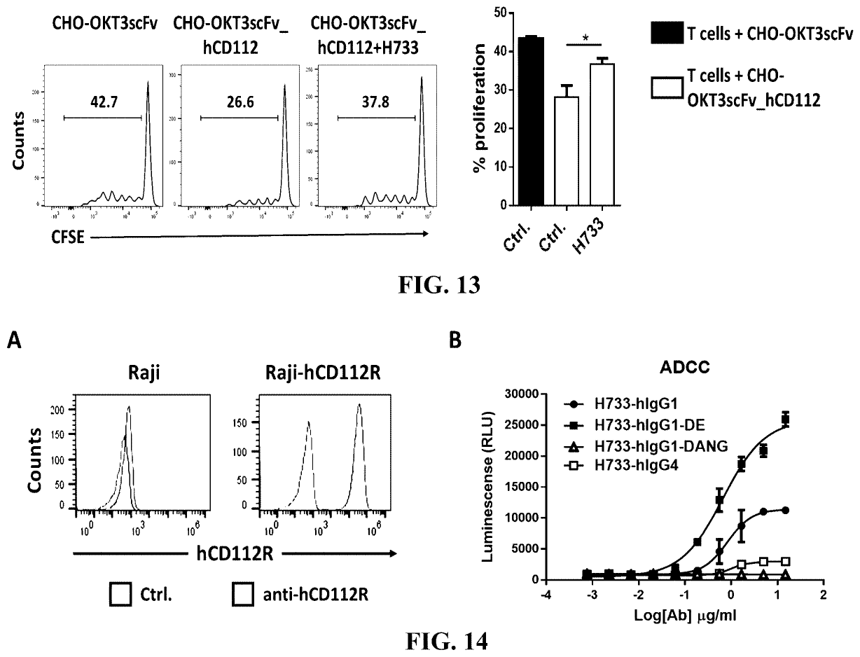

To assess the potential effects of H733 on human T cell proliferation, we labeled purified human T cells with CFSE and stimulated them with CHO-OKT3scFv or CHO-OKT3scFv-hCD112 cells. We observed a significant increase in T cell division in the presence of H733 as compared with the control group, as revealed by dilution of CFSE dye (FIG. 13). This data indicated that blockade of the CD112R-CD112 interaction by H733 could enhance the human T cell proliferation.

Taking together, these results demonstrated that H733 efficiently reversed the CD112R-mediated inhibitory functions on human NK and T cells, which suggested that H733 might function to enhance cancer cell killing by blocking CD112R-CD112 interaction in vivo.

### Example 7. H733-hIgG4 mAb Exerted No CDC Effector Function and Weak ADCC Effector Function

### 7.1 Methods

### Construction of Abs in the Context of Different Fc

Various H733 Abs in the context of different human IgG isotypes (hIgG1 and hIgG4), and three Fc variants of hIgG1 isotype: Fc-D265A/N297G (DANG) variant; Fc-S239D/1332E (DE) and Fc-S239D/A330L/1332E (DLE) variants with abolished or enhanced complement and FcγR-mediated effector functions were constructed by specific site mutations and expressed in HEK 293F cells by transient transfection.

### Complement-Mediated Cytotoxicity (CDC) Assay.

For CDC assay, Raji cells stably expressing full-length hCD112R (Raji-hCD112R) were established and used as target cells in this assay. Cells were seeded in a U-bottom 96-well plate at 2.5×10⁴ cells/well, incubated with 5 μg/ml Abs in the presence of 5% rabbit sera (Sigma). After 2 h of incubation, the supernatants in each well were analyzed for LDH release using a CytoTox 96® Non-Radioactive Cytotoxicity Assay kit (Promega).

### Antibody-Dependent Cell-Mediated Cytotoxicity (ADCC) Assay Using T Cell Lines.

For ADCC assay, Raji-hCD112R cells were used as target cells in this assay. A Jurkat cell line stably expressing human FcγRIIIa (F158) and a nuclear factor of activated T cells (NFAT)-response-element driven firefly luciferase reporter (named as Jurkat-NFAT-Luc2p/hFcγRIIIa (F158)) was generated and used as effector cells. Target cells (15000 cells/well) were seeded into the U-bottom 96-well cell culture plates and incubated briefly with different Abs. The effector cells were added (90000 cells/well) into the wells subsequently and incubated for 5 h at 37° C. in RPMI 1640 medium supplemented with 1% heat-inactivated fetal bovine serum. ADCC activity was determined by luciferase expression according to the instruction of Bright-Glo™ Luciferase assay reagents (Promega).

### 7.2 Results

Since CD112R preferentially expresses on T cells and NK cells, CD112R blocking Abs linked to naturally occurring type of IgG-Fc proteins are expected to induce Fc-mediated effector functions, such as antibody-dependent cell-mediated cytotoxicity (ADCC) and complement-dependent cytotoxicity (CDC), to different degrees depending on the IgG isotypes, which may result in the elimination of activated effector cells. Therefore, in order to prevent CD112R⁺ T cells from being killed, H733 was linked to human IgG4, and its ability to induce ADCC and CDC was evaluated by cell-based assays.

To test Abs' ADCC, a modified system was used here, in which Jurkat T lymphocyte cells expressing the human FcγRIIIa (F158) and an NFAT response element driving expression of firefly luciferase (Jurkat-NFAT-Luc2p/hFcγRIIIa (F158)) were used as effector cells, and Raji-hCD112R were used as target cells (FIG. 14A). Jurkat-NFAT-Luc2p/hFcγRIIIa (F158) cells were co-cultured with Raji-hCD112R cells in 96-well U-bottom plates in the presence of H733 with different formats for 5 h. Cytotoxicity was examined by LDH release. As expected, H733 linked to hIgG1 or hIgG1-DE variant (S239D/1332E mutations with enhanced Fc-mediated effecter function) exhibited significant ADCC. In comparison, H733 linked to hIgG4 induced weaker ADCC, and H733 linked to hIgG1-DANG variant (D265A/N297G mutations with abolished Fc-mediated effector function) did not induce ADCC (FIG. 14B).

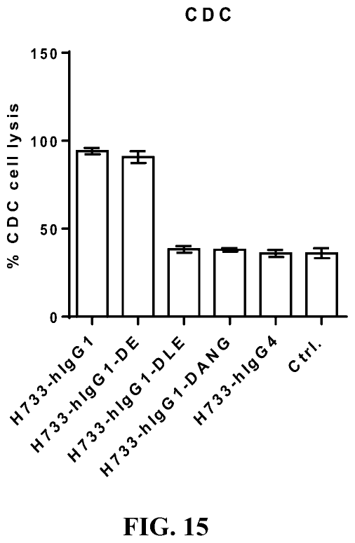

For testing Abs' CDC activities, Raji-hCD112R cells were used as target cells (FIG. 14A) and Lactate dehydrogenase (LDH) release was used as readout of target cell lysis through complements. Results showed that only H733 hIgG1 and hIgG1-DE exhibited effective CDC function, but not H733 hIgG4 and other variants (FIG. 15).

In conclusion, these cell-based assays suggested that H733-hIgG4 exerted weak Fc-mediated effecter functions as compared with H733-hIgG1 Ab. Therefore, H733 (hIgG4 form) will be used to examine the antitumor effects in vivo.

### Example 8. H733 Exerted Potent Antitumor Activity in Xenogeneic Mouse Tumor Models

### 8.1 Methods

### Xenograft Mouse Tumor Models and Treatments

For the MDA-MB231 tumor model, 1×10⁶ MDA-MB-231 cells were mixed with 3×10⁵ unstimulated PBMCs and inoculated subcutaneously (s.c.) into the right flank of 6-8 weeks old NSG mice on day 0. For the A375 tumor model, 3×10⁶ A375 cells were mixed with 1×10⁶ unstimulated PBMCs and inoculated s.c. into the right flank of 6-8 weeks old NSG mice on day 0. For the A549 tumor model, 2×10⁶ A549 cells were mixed with 6×10⁵ unstimulated PBMCs from healthy donors and inoculated subcutaneously into the right flank of NCG mice on day 0. Mice were randomized into two groups (n=3-6 per group) with similar tumor volumes (100-150 mm³): no-treatment control group or H733-hIgG4 treatment group (10 mg/kg), and followed by Ab treatment. Treatment was repeated twice weekly.

### 8.2 Results

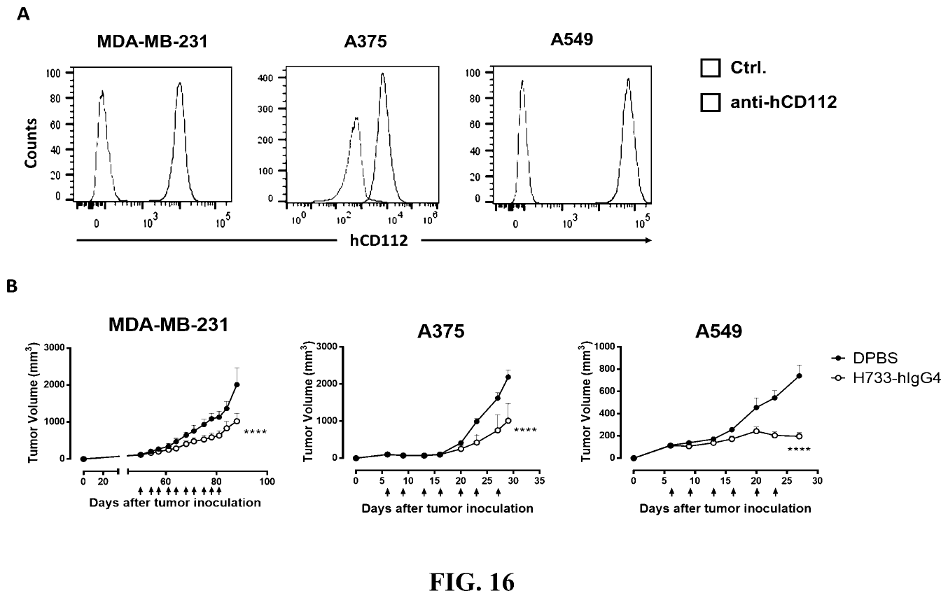

As H733 does not bind to mCD112R, we used human peripheral blood mononuclear cells (PBMCs)-based humanized xenogeneic mouse tumor models to evaluate the in vivo antitumor effects of H733. Six-8-week-old female immunodeficient NSG mice were s.c. inoculated with an admixture of human PBMCs and tumor cells (MDA-MB-231 human breast cancer cells, A375 human melanoma cells, or A549 human lung carcinoma cells; note that all of these tumor cells are known as hCD112 positive (FIG. 16A). H733 treatment resulted in tumor regressions of all these tumors as compared with the no-treatment control group (FIG. 16B). This result indicated that H733 exerted potent antitumor activity in vivo.

1. An antigen-binding protein which specifically binds to CD112R.

2. The antigen-binding protein according to claim 1 which is an antibody or antigen-binding fragment thereof.

3. The antigen-binding protein according to claim 1, comprising:
(1) an immunoglobulin heavy chain variable region comprising:
a HCDR1 as set forth in SEQ ID NO: 1 or an amino acid sequence as set forth in SEQ ID NO: 1 with addition, deletion or substitution of one or more amino acid(s),
a HCDR2 as set forth in SEQ ID NO: 2 or an amino acid sequence as set forth in SEQ ID NO: 2 with addition, deletion or substitution of one or more amino acid(s), and
a HCDR3 as set forth in SEQ ID NO: 3 or an amino acid sequence as set forth in SEQ ID NO: 3 with addition, deletion or substitution of one or more amino acid(s);
and/or
(2) an immunoglobulin light chain variable region comprising:
a LCDR1 as set forth in SEQ ID NO: 4 or an amino acid sequence as set forth in SEQ ID NO: 4 with addition, deletion or substitution of one or more amino acid(s),
a LCDR2 as set forth in SEQ ID NO: 5 or an amino acid sequence as set forth in SEQ ID NO: 5 with addition, deletion or substitution of one or more amino acid(s), and
a LCDR3 as set forth in SEQ ID NO: 6 or an amino acid sequence as set forth in SEQ ID NO: 6 with addition, deletion or substitution of one or more amino acid(s).

4. The antigen-binding protein according to claim 1, comprising:
an immunoglobulin heavy chain variable (VH) region having an amino acid sequence as set forth in SEQ ID NO: 9 or an amino acid sequence with at least 85% sequence identity to the amino acid sequence as set forth in SEQ ID NO: 9; and/or
an immunoglobulin light chain variable (VL) region having an amino acid sequence as set forth in SEQ ID NO: 10 or an amino acid sequence with at least 85% sequence identity to the amino acid sequence as set forth in SEQ ID NO: 10.

5. The antigen-binding protein according to claim 1, which is a humanized antibody or antigen-binding fragment thereof.

6. The antigen-binding protein according to claim 5, wherein the humanized antibody or antigen-binding fragment thereof comprises:
(1) a heavy chain variable (VH) region comprising:
a HCDR1 having an amino acid sequence as set forth in NYLIE,
a HCDR2 having an amino acid sequence as set forth in VINPGHGFTNYX₁X₂KFX₃G, and
a HCDR3 having an amino acid sequence as set forth in GEWDWYFDV;
and/or
(2) a light chain variable (VL) region comprising:
a LCDR1 having an amino acid sequence as set forth in KASQNVGTAVA,
a LCDR2 having an amino acid sequence as set forth in STSNRYT, and
a LCDR3 having an amino acid sequence as set forth in QQX₄SSYPFT,
wherein,
X₁ is selected from A or N,
X₂ is selected from E or Q,
X₃ is selected from K or Q, and/or
X₄ is selected from C or S.

7. The antigen-binding protein according to claim 5, wherein the humanized antibody or antigen-binding fragment thereof comprises:
(1) a heavy chain variable (VH) region comprising:
a HCDR1 having an amino acid sequence as set forth in SEQ ID NO:1,
a HCDR2 having an amino acid sequence as set forth in SEQ ID NO: 2, and
a HCDR3 having an amino acid sequence as set forth in SEQ ID NO: 3;
or
a HCDR1 having an amino acid sequence as set forth in SEQ ID NO:1,
a HCDR2 having an amino acid sequence as set forth in SEQ ID NO: 7, and
a HCDR3 having an amino acid sequence as set forth in SEQ ID NO: 3
and/or
(2) a light chain variable (VL) region comprising:
a LCDR1 having an amino acid sequence as set forth in SEQ ID NO: 4,
a LCDR2 having an amino acid sequence as set forth in SEQ ID NO: 5, and
a LCDR3 having an amino acid sequence as set forth in SEQ ID NO: 6;
or
a LCDR1 having an amino acid sequence as set forth in SEQ ID NO: 4,
a LCDR2 having an amino acid sequence as set forth in SEQ ID NO: 5, and
a LCDR3 having an amino acid sequence as set forth in SEQ ID NO: 8.

8. The antigen-binding protein according to claim 5, wherein the humanized antibody or antigen-binding fragment thereof comprises:
a heavy chain variable (VH) region having an amino acid sequence as set forth in SEQ ID NO: 11 or 13, or an amino acid sequence with at least 85% sequence identity to the amino acid sequence as set forth in SEQ ID NO: 11 or 13; and/or
a light chain variable (VL) region having an amino acid sequence as set forth in SEQ ID NO: 12 or 14, or an amino acid sequence with at least 85% sequence identity to the amino acid sequence as set forth in SEQ ID NO: 12 or 14.

9. The antigen-binding protein according to claim 1, which specifically binds to the extracellular domain (ECD) of CD112R.

10. A composition comprising the antigen-binding protein according to claim 1 and a pharmaceutically acceptable carrier.

11. The composition according to claim 10, further comprising an additional therapeutic agent.

12. A nucleic acid molecule encoding the antigen-binding protein according to claim 1.

13. A host cell comprising the antigen-binding protein according to claim 1, wherein the host cell comprises a prokaryotic or a eukaryotic cell.

14. A method for prevention or treatment of a subject with a disease associated with CD112R, comprising administrating to the subject a therapeutically effective amount of the antigen-binding protein according to claim 1.

15. The method according to claim 14, wherein the disease comprises cancer, an infectious disease, sepsis, an autoimmune condition, and/or an undesirable immune activation that follows gene therapy.

16. The antigen-binding protein according to claim 1, which specifically binds to amino acids 90-150 of CD112R.

17. A host cell comprising the nucleic acid molecule according to claim 12, wherein the host cell comprises a prokaryotic or a eukaryotic cell.

18. A method for prevention or treatment of a subject with a disease associated with CD112R, comprising administrating to the subject a therapeutically effective amount of the composition according to claim 10.

19. The method according to claim 18, wherein the disease comprises cancer, an infectious disease, sepsis, an autoimmune condition, and/or an undesirable immune activation that follows gene therapy.
